(19) United States
(12) Patent Application Publication
(10) Pub. No.: US 2002/0087176 A1
(43) Pub. Date: Jul. 4, 2002

(54) Anastomosis device

(76) Inventor: E. Skott Greenhalgh, Wyndmoor, PA (US)

Correspondence Address: John A. Chionchio, Esq., Synnestvedt & Lechner LLP, Suite 2600, 1101 Market Street, Philadelphia, PA, 19107-2950, US

(21) Appl. No.: 09/974,324

(22) Filed: Oct. 10, 2001

Related U.S. Application Data
(60) Provisional application No. 60/238,914, filed on Oct. 10, 2000.

(51) Int. Cl. A61F 2/06

(52) U.S. Cl. 606/155; 623/1.15

(57) ABSTRACT

A tubular support for supporting vessels made of living tissue in an anastomosis procedure is disclosed. The support is biased so as to resiliently and elastically maintain a predetermined length and diameter. The support has ends insertable into the lumens of the vessels through openings, either in the end or in an incision in the vessel sidewall. The ends are compressed radially to a smaller diameter upon insertion and expand due to the biasing force to engage each vessel and support them in proximity to one another for surgically joining, for example, by suturing. The tubular support is formed by braiding elastic filamentary members into a tubular configuration under tension to provide the biasing force or by braiding resilient filamentary members into a tubular configuration. The tubular support may be made of bio-absorbable material or it may have an elastic, fluid impermeable membrane.

### RELATED APPLICATION

This application is based on and claims priority of U.S. Provisional Application No. 60/238,914, filed Oct. 10, 2000.

### FIELD OF THE INVENTION

This invention relates to a device used to join two vessels together, and especially to surgically join vessels such as veins and arteries within a living body.

### BACKGROUND OF THE INVENTION

Many surgical procedures involve anastomosis, the joining together of two vessels comprising living tissue within a living body. Blood vessels may be joined, for example, in an anastomosis arteriolovenularis, wherein an artery is directly connected to a vein to act like a shunt to bypass a capillary bed. There are also hetrocladic and homocladic anastomoses, which connect branches of different arteries together and branches of the same artery together, respectively.

Vessels comprising living tissue other than veins and arteries are also connected in various procedures. For example, in an ileorectal anastomosis, the ileum is connected to the rectum after total colectomy, as is often done in treatment of ulcerative colitis. Intestinal anastomosis provides for the establishment of a communication between two portions of the intestinal tract. The Rouxen-Y anastomosis is a Y-shaped anastomosis in which the small intestine segment is divided and the distal end is implanted into another organ, such as the stomach or esophagus. The proximal end is attached to the small intestine to provide drainage without reflux.

Anastomosis involves several different types of surgical connections. The simplest is the end-to-end connection, shown in FIG. 1, wherein the vessels to be joined are placed end-to-end and sutured together circumferentially around the vessel walls. More complicated is the end-to-side connection, shown in FIG. 2, wherein a first vessel having a free end is sutured to the sidewall of a second vessel about an opening in the second vessel.

Significant surgical skill is required to perform anastomosis procedures. In particular, it is difficult to join the vessels over the entire perimeter of the openings while maintaining the lumen of each vessel fully open and aligned. The vessels, made of living tissue, tend to be extremely flexible and deflect under the slightest force, making it difficult to maintain an unchanging positional relationship between the vessel walls during suturing. The walls will shift relatively to one another and collapse inwardly into the lumen during surgery, presenting complications to the surgeon. The difficulties are more problematic the smaller the vessels that are to be joined.

There is clearly a need for a device which will make anastomosis easier for the surgeon and avoid the difficulties associated with joining two extremely flexible tubes together.

### SUMMARY OF THE INVENTION

The invention concerns a tubular support having an end insertable within a lumen of a vessel comprising living tissue. The tubular support comprises a plurality of elastic filamentary members interlaced by braiding, the elastic filamentary members being braided under a predetermined tension so as to provide a biasing force elastically maintaining the tubular support at a predetermined length and a predetermined diameter. Being braided, the filamentary members are movable relatively to one another allowing the tubular support to be elastically radially compressible upon application of a radial compressive force. Under such a force, the tubular support is compressible to a relatively smaller diameter, thereby adapting the end for insertion into the lumen of the vessel. Because the members are elastic and braided under tension, the tubular support is elastically self expanding and will go back to the predetermined diameter upon removal of the compressive force to engage and support the vessel.

Preferably, the tubular support has another end insertable within a lumen of another vessel comprising living tissue. When the ends are inserted into the lumens of the respective vessels, the tubular support engages and holds the vessels in proximity to one another for attaching the vessels together in fluid communication with one another.

The tubular support may further comprise an elastic membrane attached lengthwise along itself. The membrane has a relatively low permeability to fluids allowing the tubular support to act as a conduit between the vessels.

The tubular support may have several configurations. For example, it may have a bend located between the ends thereby arranging the ends non co-linearly relatively to one another and adapting the tubular support to extend between the vessels which are oriented angularly with respect to one another. In such a configuration, one of the ends is insertable into the lumen through an incision in a sidewall of the respective vessel, the other of the ends extending outwardly from the incision for engagement with the lumen of the other vessel in an end-to-side anastomosis. Preferably, an aperture is positioned in the tubular support at the bend thereby allowing fluid flow through both the vessels.

Alternatively, the tubular support may comprise a plurality of resilient filamentary members interlaced by braiding. The filamentary members are biasable to resiliently maintain the tubular support at a predetermined diameter and a predetermined length. Being braided, the filamentary members are movable relatively to one another allowing the tubular support to be resiliently radially compressible upon application of a radial compressive force to a relatively smaller diameter, thereby adapting the respective ends for insertion into respective lumens of the two vessels to be joined together. The tubular support is biased by the filamentary members to resiliently expand upon removal of the compressive force to engage and support the vessels in proximity for attachment to one another. Preferably, an elastic membrane is attached lengthwise along the tubular support, the membrane having a relatively low permeability to fluids to allow the tubular support to act as a fluid conduit between the first and the second vessels.

The invention also contemplates a method of joining together two vessels comprising living tissue, each having a respective lumen. The method comprises the steps of:

(a) providing an elongated tubular support according to one of the embodiments described above;

(b) creating a respective opening in each of the vessels;

(c) compressing the tubular support radially to a diameter sized to fit within the openings of the vessels;

(d) inserting one end of the tubular support into the lumen of one of the vessels through the respective opening in the one vessel;

(e) inserting the other end of the tubular support into the lumen of the other of the vessels through the respective opening in the other of the vessels; and

(f) discontinue compressing the tubular support radially, thereby allowing the tubular support to expand to the predetermined diameter, the tubular support thereby engaging and supporting each of the vessels.

If the tubular support has a fluid impermeable membrane, the support is attached to the vessel and acts as a conduit connecting the vessels in fluid communication. However, if the vessels are to be directly attached to one another, the method further includes the steps of bringing the respective openings adjacent to one another and attaching the vessels to each other at the openings. Preferably, the vessels are attached by suturing.

It is an object of the invention to provide a tubular support insertable into the lumen of a vessel for supporting the vessel and attaching it to another vessel.

It is another object of the invention to provide a tubular support for attaching flexible vessels in end-to-end or end-to-side anastomoses.

It is still another object of the invention to provide a tubular support which can serve as a fluid impermeable conduit providing fluid communication between the lumens of vessels.

It is again another object of the invention to provide a tubular support permitting vessels to be sutured together.

These and other objects and advantages of the invention will be apparent upon consideration of the following drawings and detailed description of the preferred embodiments.

### BRIEF DESCRIPTION OF THE DRAWINGS

FIG. 1 shows two vessels being joined in an end-to-end anastomosis;

FIG. 2 shows a side-to-end anastomosis of two vessels;

FIG. 3 shows a side view of an embodiment of the anastomosis device according to the invention;

FIG. 4 shows a side view of an alternate embodiment of an anastomosis device according to the invention;

FIG. 5 shows a side view of another alternate embodiment of an anastomosis device;

FIG. 6 shows a sectional view of an anastomosis device joining two vessels;

FIGS. 7-9 are sectional views of an anastomosis device being implanted to effect the joining of two vessels;

FIG. 10 shows a sectional view of an anastomosis device being used to couple two vessels together;

FIG. 11 shows a side view of an alternate embodiment of an anastomosis device;

FIG. 12 shows a side view of an anastomosis device useable in an end-to-side anastomosis;

FIG. 13 shows an end view of the anastomosis device shown in FIG. 12 taken along lines 13-13; and

FIG. 14 shows the anastomosis device of FIG. 12 used in an end-to-side anastomosis.

### DETAILED DESCRIPTION OF THE PREFERRED EMBODIMENTS

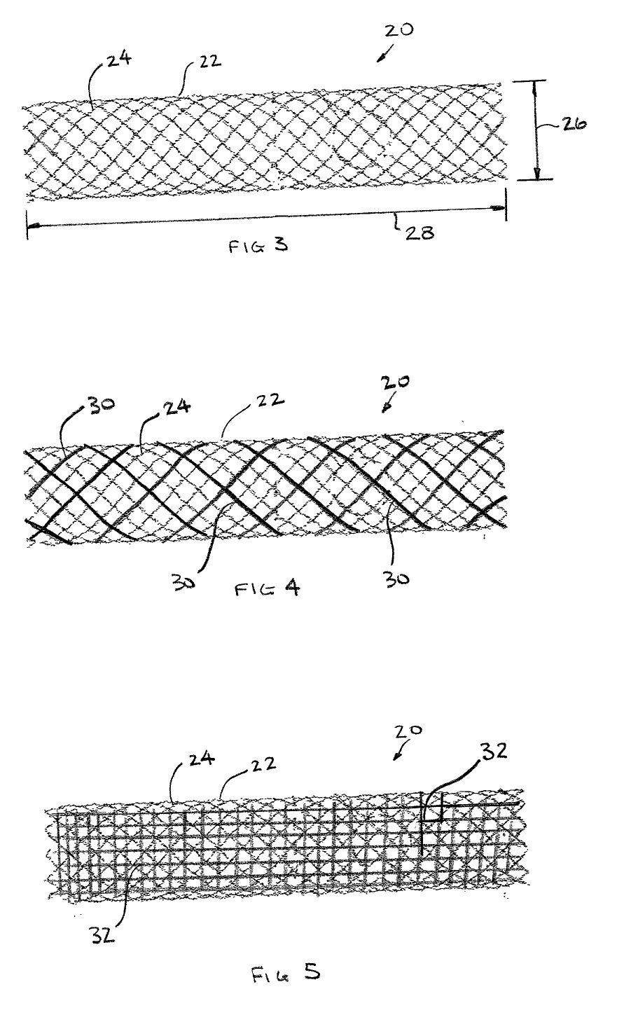

FIG. 3 shows an anastomosis device 20 according to the invention which comprises a tubular support 22 formed of a plurality of interlaced filamentary members 24. Filamentary members 24 are preferably interlaced by braiding to take advantage of the “trellis effect” associated with braided structures. The trellis effect refers to the characteristic of a braided tube to contract in diameter when the tube is stretched lengthwise by a tensile force and expand in diameter when the tube is subjected to a lengthwise-applied compression force shortening the tube. The braided tube also displays complementary behavior in that, if the diameter of the tube is contracted by application of an externally applied radial force, the tube will lengthen, and if the diameter of the tube is expanded radially outwardly, the tube will shorten in response.

The tubular support 22 has biasing means which causes it to assume a nominal diameter 26 and length 28 when not subjected to any external forces. Various biasing means are feasible. For example, filamentary members 24 may be made of an elastic material such as spandex, rubber or texturized filaments. The braiding is performed under tension so as to provide a biasing force using the elastic nature of the material to elastically maintain the tubular support at a desired predetermined length and diameter. Alternatively, the filamentary members 24 may be made of a resilient material, such as nitinol, elgiloy or stainless steel. When any of these materials are braided, they are strain hardened to assume a particular shape and tend to develop internal elastic forces which will return the tubular support 22 to the nominal diameter 28 and length 28 at which the filamentary members were originally braided. To best control the nominal diameter, it is advantageous to braid and strain harden the filamentary members over a mandrel having the desired diameter.

Another biasing means comprises incorporating elastic filamentary members 30 in the tubular support as shown in FIG. 4. Again, elastic material such as spandex, rubber and highly texturized PGA, PLA or other yarns may be used. The elastic filamentary members will stretch when the tubular support is stretched lengthwise, but once the stretching force is removed, the elastic filamentary members will contract to their unstressed length and tend to pull the tubular support back to its original length and diameter. The elastic filamentary members 30 may be positioned internally or externally of the support but are preferably interbraided with the resilient filamentary members 24 forming the support as shown in FIG. 4. In conjunction with resilient filamentary members 24, the elastic filamentary members 30 may be interbraided under a tension force which causes them to contract once the tension force is released after braiding. This tends to bias the tubular support 22 into a shorter length of greater diameter.

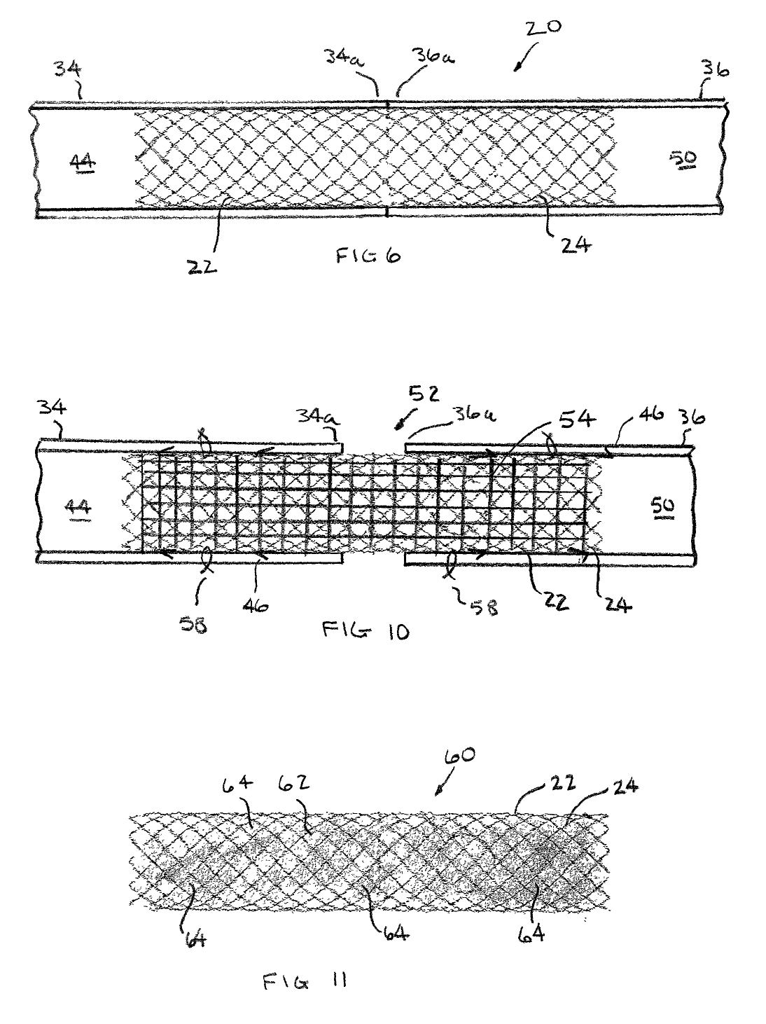

Biasing may also be provided by an elastic membrane 32 shown in FIG. 5. The membrane may be formed by an elastic material, such as silicone, polyurethane or elastic film which is applied as a continuous coating over the tubular support 22. The membrane may also be a braided, knitted or woven sleeve, affixed externally, internally or interlaced with other filamentary members forming the tubular support. The elasticity of the membrane allows the tubular support to be stretched and compressed, as described above, but biases it back into its nominal diameter and length in the absence of external forces.

In its simplest embodiment, shown in FIG. 6, the anastomosis device 20 forms an elastic, resilient frame or stent which supports the vessels 34 and 36 to be joined in a convenient end-to-end positional relationship which allows them to be sutured or stapled together at their respective ends 34a and 36a to form the appropriate connection.

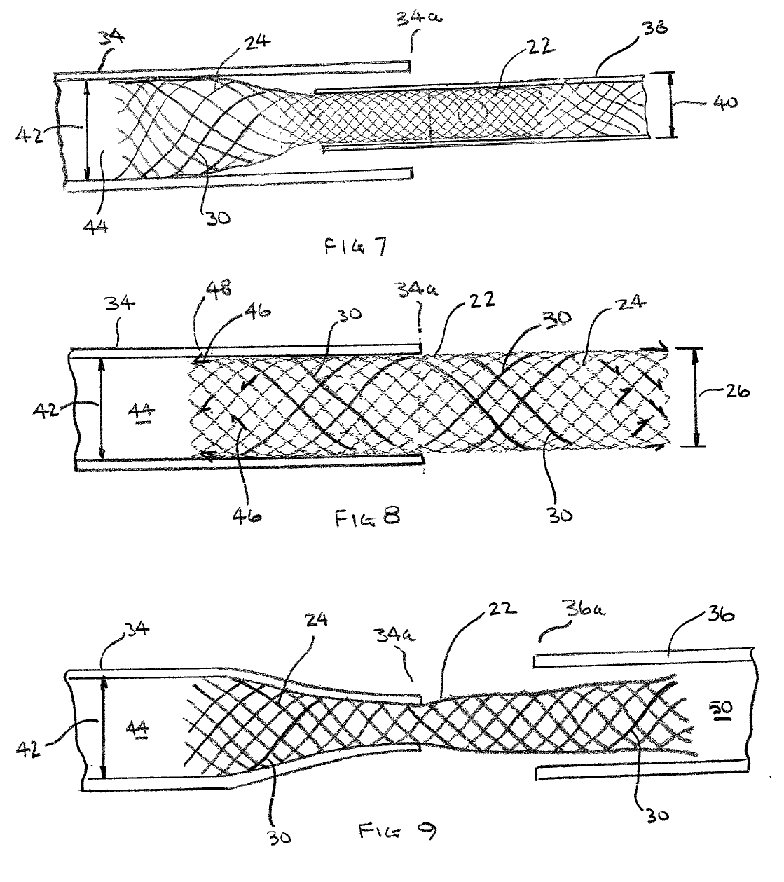

FIGS. 7-9 show how the device is used. Preferably, the tubular support 22, having a nominal diameter appropriate for the vessels 34 and 36 to be joined, is prepackaged in a rigid tube 38. Tube 38 has a smaller outer diameter 40 than the inner diameter 42 of lumen 44 in vessel 34. Due to its braided structure, support 22 can be radially collapsed to fit within tube 38. As shown in FIG. 7, tube 38 is inserted into lumen 44 of vessel 34 and the tubular support 22 is forced out of the tube 38 while the tube is withdrawn from the lumen 44. Due to its biasing means 30, the tubular support 22 expands radially as it exits tube 38, substantially reaching its full nominal diameter 26 once entirely free of the tube, as shown in FIG. 8. Nominal diameter 26 is preferably sized to have a slightly larger diameter than lumen 44, allowing for an interference engagement between the tubular support 22 and the vessel 34, thereby securing the device to the vessel. If a more positive engagement is necessary, hooks 46 may be positioned on the support which dig into the vessel and anchor the tubular support 22 in place. The hooks are oriented with their piercing ends 48 facing toward the open end of vessel 34 to allow movement of the support further into the vessel 34 but prevent outward movement.

As shown in FIG. 9, the vessel 34 with the tubular support 22 is compressed in diameter. This may be accomplished by hand or by a tool which imparts a radially compressive force on the vessel and support. Due to the trellis effect, the tubular support 22 readily collapses and lengthens allowing the end of the support extending from the vessel 34 to be inserted into the lumen 50 of vessel 36. When the ends 34a and 36a of each vessel are in or near to abutting relationship as shown in FIG. 8, the compression of support 22 and vessel 34 is released, and the biasing means 30 of the tubular support 22 expands the support back to its nominal diameter, thereby causing the support to engage vessel 36. The support also shortens as it expands radially outwardly to draw the vessel ends 34a and 36a together. The butted ends may now be sutured or stapled, the tubular support 22 holding the vessels securely together end-to-end during the surgery and preventing the vessels from collapsing, moving out of alignment or pulling apart while being worked on.

When used in the manner described above to position, hold and support the vessels during surgery, it is preferable to make the tubular support 22 from a biodegradable material such as polyglycolic acid, polylactic acid or some combination thereof. A tubular support made of these substances will be absorbed and eliminated by the natural reaction of the body to foreign substances. Once the vessels heal together, there should no longer be any need for the support. Thus, it is advantageous to have it degrade over time and eventually disappear after it has served its purpose during the surgery.

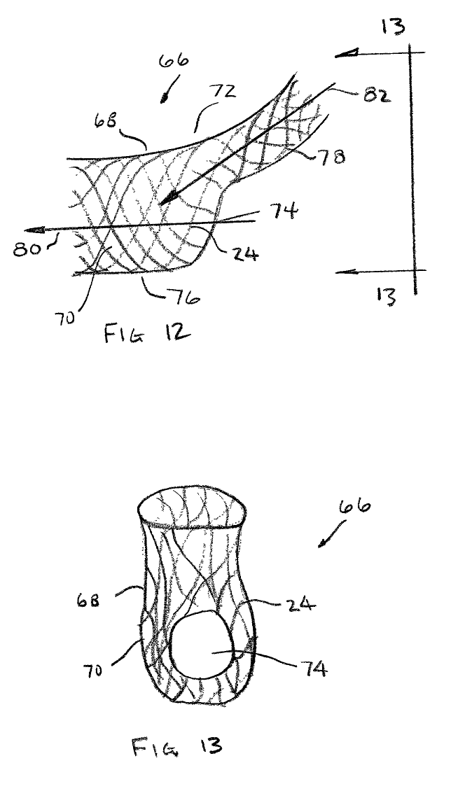

An alternate embodiment 52 of the anastomosis device is shown in FIG. 10. Device 52 comprises the tubular support 22 formed by the interlaced filamentary members 24, the device also comprising a membrane 54 sealing the support 22 substantially over its length. The alternate embodiment 52 forms a sealing connection between the vessels 34 and 36 and does not require that the ends of the vessels 34a and 36a be in proximity for surgical joining by sutures or staples. The device acts as a bridge which sealingly joins the lumens 44 and 50 in fluid communication and replaces a portion of the vessel.

To function effectively, membrane 54 must have low permeability so as not to allow fluids to leak at the joint. For blood vessels, a permeability of at most 350 cc/min/cm² is feasible. The membrane should also be relatively flexible and elastic so as to permit the device to be installed as described above and also provide flexibility compatible with the surrounding tissue allowing the vessel to bend and flex at the joint. All of the material comprising the anastomosis device 52 must be biocompatible.

A preferred construction for the membrane 54 comprises polyester yarns of relatively small diameter, on the order of 100 denier, braided at a density of 150 picks or yarns per inch of axial length to form a sleeve with the required permeability. Polyester is preferred for its biocompatibility and successful history in human implants. It is advantageous to form the membrane from relatively small yarns tightly spaced. This provides for small interstices between the yarns, yielding the relatively low permeability required and also results in a thin-walled membrane which is relatively flexible and not bulky, allowing the device to be collapsed into a relatively small diameter for insertion into relatively small diameter vessels.

Filamentary members 24 are preferably formed of 0.003 diameter nitinol wire due to its biocompatibility, excellent flexibility and great resilience. Nitinol is formable over a mandrel to a nominal shape and diameter, the filamentary members being readily strain hardenable and providing the biasing means which allows the device to be easily collapsed when under a tensile or radial force, but then expanding back to the nominal diameter and shape once the distorting force is removed. Preferably, the filamentary members 24 are interbraided with the polyester yarns forming the membrane 54 to provide an integral structure which joins and seals the vessels together. Alternatively, the membrane 54 could be positioned over the outside or lining the inside of the tubular support 22 and sutured or adhesively bonded thereto.

While it is preferred to form the membrane 54 by braiding, it may also be possible to interlace the yarns by knitting or weaving. Braiding is preferred because it provides the expandability due to the trellis effect described above, which allows the device to be collapsed and expanded without significant wrinkling or folding occurring.

Anastomosis device 52 is intended to be a permanent implant and, therefore, should be securely fastened to the vessels 34 and 36. This may be accomplished by means of hooks 46 described above and shown in FIG. 8 or by sutures 58 shown in FIG. 10. Suturing of the device in place should proceed much easier because the device supports the vessels with the lumens wide open and prevents collapse of the vessel walls during the procedure. As a permanent implant, it is also desirable that the materials from which the device is formed promote the inter-growth of tissue, such as endothelial cells, with the device. This will help to seal the device over time and possibly promote the joining of the vessels 34 and 36 by tissue rather than an artificial device, which could eventually decay and need replacement.

FIG. 11 shows yet another alternate embodiment 60 of the anastomosis device according to the invention wherein the tubular support 22 comprises filamentary members 24 of braided nitinol wires which provide the support and biasing means for the device, and a membrane 62 made of a silicone coating which seals the interstices 64 between the filamentary members 24. Device 60 is intended to be used as a permanent implant in a similar fashion to device 52. The silicone provides a flexible membrane with virtually zero permeability to sealingly join the vessels together. The nitinol and silicone are both biocompatible materials which should prove to be a successful combination in the device.

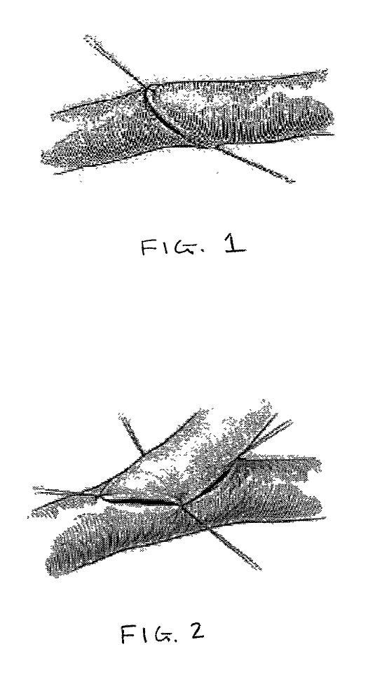

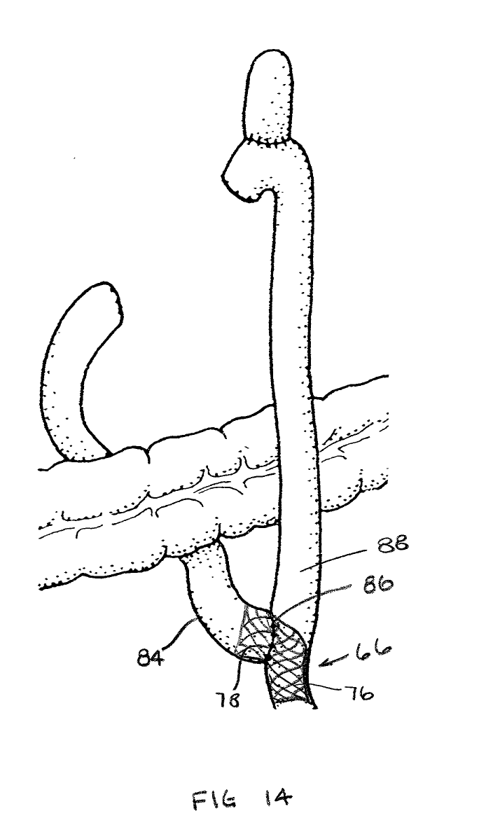

The embodiments of the anastomosis devices, thus, far described are intended for use in the end-to-end vessel connection of FIG. 1. FIGS. 12 and 13 show an anastomosis device 66 according to the invention which may be used to effect the end-to-side connection of FIG. 2. Such a connection is used in the Rouxen-Y anastomosis, described in the background section above and illustrated in FIG. 14.

Device 66 in its simplest embodiment comprises filamentary members 24, preferably of nitinol and preferably braided to form a tube 68, having a tube wall 70 and a bend 72. An aperture 74 is formed in the tube wall at the bend 72 along the outside curve of the tube 68. Together, the aperture and the bend define a main tube portion 76 and a branch tube portion 78. There are, thus, two possible paths for the relative unimpeded flow of fluid through the device, one path through aperture 74 and into the main tube portion 76, indicated by arrow 80 and another path through branch tube portion 78 and into main tube portion 76, indicated by arrow 82.

Device 66 may be formed by braiding filamentary members 24 around a kinked mandrel to establish the bent shape, the filamentary members, when made of nitinol, being work hardened by the braiding process and remaining nominally in the kinked shape after removal of the tube from the mandrel. Alternatively, if the filamentary members 24 are made of another material, for example, a polymer such as polyester, or polypropylene or a biodegradable material such as polyglycolic acid, the material should be permanently settable into the desired shape by means of heat or chemical processes to bias the filamentary members to remain in the bent shape after removal from the mandrel.

Due to the braided structure, aperture 74 can be easily formed in the tube wall 70 without cutting any filamentary members by merely separating the filamentary members outwardly. The filamentary members may be set or biased into the open position by strain hardening, heat or chemical means as appropriate.

As shown in FIG. 14, the device 66 may be used without a membrane as a support structure to align and hold the proximal end of the divided small intestine support 84 in contact with the opening 86 in the side of the small intestine 88. The small intestines may then be sutured or stapled together to complete the anastomosis.

Device 66 may also comprise a membrane (not shown) as described above to permit it to sealingly couple vessels together without the need for actually joining the vessel tissue directly. The device 66 may be sutured, stapled or attached with hooks as described in the earlier embodiments. The membrane is preferably braided yarns but could also be knitted, woven or an elastic coating.

The anastomosis device according to the invention should allow surgical connections between vessels to be made with greater ease, reliability and success. The device may also promote faster post operative healing, result in fewer complications, shorter hospital stays for patients and generally improve the state of the art in anastomosis procedures.

What is claimed is:

1. A tubular support having an end insertable within a lumen of a vessel comprising living tissue, said tubular support comprising a plurality of elastic filamentary members interlaced by braiding, said elastic filamentary members being braided under a predetermined tension so as to provide a biasing force elastically maintaining said tubular support at a predetermined length and a predetermined diameter, said filamentary members being movable relatively to one another allowing said tubular support to be elastically radially compressible upon application of a radial compressive force to a relatively smaller diameter thereby adapting said end for insertion into the lumen of said vessel, said tubular support being elastically self expanding to said predetermined diameter upon removal of said compressive force to engage and support said vessel.

2. A tubular support according to claim 1, further comprising another end insertable within a lumen of another vessel comprising living tissue, said tubular support engaging and holding said vessels in proximity to one another for attaching said vessels together in fluid communication with one another.

3. A tubular support according to claim 2, wherein said filamentary members comprise spandex.

4. A tubular support according to claim 2, wherein said filamentary members comprise a material absorbable in living tissue.

5. A tubular support according to claim 4, wherein said filamentary members comprise texturized filamentary members of polyglycolic acid.

6. A tubular support according to claim 4, wherein said filamentary members comprise texturized filamentary members of polylactic acid.

7. A tubular support according to claim 1, further comprising a plurality of hooks each having an end projecting angularly outwardly from said tubular support to engage said vessel, said ends facing toward the middle of said tubular support.

8. A tubular support according to claim 2, further comprising an elastic membrane attached lengthwise along said tubular support, said membrane having a relatively low permeability to fluids allowing said tubular support to act as a conduit between said vessels.

9. A tubular support according to claim 8, wherein said elastic membrane comprises a substantially continuous elastic coating, said filamentary members being embedded within said coating, said coating sealing interstices between said filamentary members and thereby establishing said relatively low permeability.

10. A tubular support according to claim 9, wherein said coating comprises silicone.

11. A tubular support according to claim 2, further comprising:
a bend located between said ends thereby arranging said ends non co-linearly relatively to one another and adapting said tubular support to extend between said vessels when oriented angularly with respect to one another, one of said ends being insertable into said lumen through an incision in a sidewall of said one vessel, the other of said ends extending outwardly from said incision for engagement with said lumen of said other vessel; and
an aperture positioned in said tubular support at said bend thereby allowing fluid flow through both said one vessel and said other vessel.

12. A tubular support having first and second ends insertable within respective lumens of first and second vessels comprising living tissue for joining said vessels in fluid communication, said tubular support comprising:
a plurality of resilient filamentary members interlaced by braiding, said filamentary members being biasable to resiliently maintain said tubular support at a predetermined diameter and a predetermined length, said filamentary members being movable relatively to one another allowing said tubular support to be resiliently radially compressible upon application of a radial compressive force to a relatively smaller diameter thereby adapting said respective ends for insertion into said lumens of said first and second vessels, said tubular support being biased by said filamentary members to resiliently expand upon removal of said compressive force to engage and support said vessels in proximity for attachment to one another; and
an elastic membrane attached lengthwise along said tubular support, said membrane having a relatively low permeability to fluids allowing said tubular support to act as a fluid conduit between said first and said second vessels.

13. A tubular support according to claim 12, wherein said resilient filamentary members comprise nitinol.

14. A tubular support according to claim 12, wherein said elastic membrane comprises a plurality of elastic filamentary members braided together to form a sleeve surrounding said tubular support.

15. A tubular support according to claim 14, wherein said elastic filamentary members comprise polyester.

16. A tubular support according to claim 14, wherein said elastic filamentary members are interbraided with said resilient filamentary members.

17. A tubular support according to claim 12, wherein said elastic membrane comprises a substantially continuous elastic coating, said resilient filamentary members being embedded within said coating, said coating sealing interstices between said resilient filamentary members and thereby establishing said relatively low permeability.

18. A tubular support according to claim 17, wherein said coating comprises silicone.

19. A tubular support according to claim 12, further comprising a plurality of elastic filamentary members interbraided with said resilient filamentary members, said elastic filamentary members being interbraided under tension to provide said biasing force for biasing said tubular support and thereby maintaining it at said predetermined diameter and length.

20. A tubular support according to claim 12, further comprising:
a bend located between said first and second ends thereby arranging said ends non co-linearly relatively to one another and adapting said tubular support to extend between said first and second vessels when oriented angularly with respect to one another, one of said ends being insertable into said lumen of said first vessel through an incision in a sidewall of said first vessel, the other of said ends extending outwardly from said incision for engagement with said lumen of said second vessel; and
an aperture positioned in said tubular support at said bend thereby allowing fluid flow through both said first and said second vessel.

21. A method of joining together two vessels comprising living tissue, each said vessel having a respective lumen, said method comprising the steps of:
providing an elongated tubular support having first and second ends insertable into respective lumens of said vessels, said tubular support comprising a plurality of flexible filamentary members interlaced by braiding, said filamentary members being biased to maintain said tubular support at a predetermined length and a predetermined diameter, said filamentary members being flexibly movable relatively to one another thereby allowing said tubular support to be radially compressible upon application of a radial compressive force to a relatively smaller diameter thereby adapting said ends for insertion into said respective lumens of said vessels, said tubular support being biased to expand to said predetermined diameter upon removal of said compressive force;
creating a respective opening in each of said vessels;
compressing said tubular support radially to a diameter sized to fit within the openings of said vessels;
inserting one end of said tubular support into the lumen of one of said vessels through said respective opening in said one vessel;
inserting the other end of said tubular support into the lumen of the other of said vessels through said respective opening in the other of said vessels; and
discontinue compressing said tubular support radially thereby allowing said tubular support to expand to said predetermined diameter, said tubular support thereby engaging and supporting each of said vessels.

22. A method according to claim 21, further comprising the steps of bringing said respective openings adjacent to one another, and attaching said vessels to each other at said openings.

23. A method according to claim 22, wherein said vessels are attached by suturing.
